(19) United States
(12) Patent Application Publication
(10) Pub. No.: US 2020/0048714 A1
(43) Pub. Date: Feb. 13, 2020

(54) Method and device for acquiring fetal fraction of cell-free DNA, storage medium and electronic device

(71) Applicant: Beijing USCI Medical Devices Co.,Ltd., Beijing (CN)

(72) Inventors: Yongtao GUAN, Beijing (CN); Hanli XU, Beijing (CN); Jingbo ZHANG, Beijing (CN); Weiwei Wang, Beijing (CN); Qixi WU, Beijing (CN); Nan FANG, Beijing (CN); Ling BAI, Beijing (CN); Jianwei WANG, Beijing (CN)

(21) Appl. No.: 16/587,922

(22) Filed: Sep. 30, 2019

(30) Foreign Application Priority Data
Sep. 30, 2018 (CN) 201811162012.9

(51) Int. Cl. C12Q 1/6883; G16B 30/00; C12Q 1/6809; C12Q 1/6858; G16B 40/10; C12Q 1/686; C12Q 1/6827; G06F 17/18; G16H 50/50; G16H 50/30

(52) U.S. Cl. CPC C12Q 1/6883; G16B 30/00; C12Q 1/6809; C12Q 1/6858; G16B 40/10; C12Q 2600/118; C12Q 1/6827; G06F 17/18; G16H 50/50; G16H 50/30; C12Q 2600/154; C12Q 1/686

(57) ABSTRACT

Disclosed are a method and a device for acquiring the fetal fraction of cfDNA, a storage medium and an electronic device. The method comprises: acquiring sequencing data of a sample taken from a mother pregnant with a fetus; establishing a joint probability distribution model of the maternal and fetal genotypes, the joint probability distribution model containing one or more factors affecting the read heterozygosity, the percentage of the read heterozygosity being the ratio of the number of SNPs covered by different bases to the total number of SNPs covered by more than one reads in the sequencing data; substituting the values of the one or more factors and of the acquired read heterozygosity into the established joint probability distribution model; and obtaining the fetal fraction of cfDNA by maximum likelihood estimation of the joint probability distribution model.

### TECHNICAL FIELD

The disclosure relates to the field of biological detection, and in particular to a method and a device for acquiring the fetal fraction (FF) of cell-free DNA (cfDNA), a storage medium and an electronic device.

### BACKGROUND

The quantification of FF of cfDNA plays an important role in noninvasive prenatal screening (NIPS), and it determines how effective is the NIPS. The significance of FF inference is reflected in: First, in the case of a known FF, for a sample with a very low FF (for example, less than 3%), a report of ‘inconclusive’ is provided, and it is suggested that the pregnant woman should choose another method for prenatal testing. In this way, false negatives of the NIPS can be avoided to a great extent, as an excessively low FF is a major cause of false negatives. Second, in the case of a known FF, the expected chromosomal dosage change for fetal aneuploidy is known, and the statistical power of NIPS can be improved. Third, in the case of a known FF, NIPS for special samples with aneuploidy in sex chromosomes, or with twin pregnancy, or aneuploidy with mosaicism, or the likes become easier, with higher accuracy. Thus, how to accurately quantify FF is an important problem to be solved.

Current available quantification methods for FF include:

(1) Real-Time Quantitative PCR Technology

In 1998, Denis Lo et al., working in the Chinese University of Hong Kong, quantitatively analyzed fetal cfDNA in maternal plasma using real-time quantitative PCR technology, and discovered that fetal cfDNA can be detected as early as 7th gestational week, and the fraction increases along with the increase of gestational weeks. Taking the real-time fluorescence quantitative PCR method for example, primers were designed to amplify and detect the sex determining region Y (SRY) gene in a maternal peripheral plasma sample. Such methods are based on that the SRY is a marker gene of a male fetus, and does not exist in the maternal cfDNA. According to a standard curve, the copy number of SRYs in the sample per milliliter was estimated, thus the FF of the male fetus was inferred.

(2) Whole-Genome Next-Generation Sequencing (NGS), FF Inference Based on Sex Chromosomes

On the basis of high-throughput NGS, whole-genome low-depth sequencing data of maternal peripheral plasma could be obtained in NIPS. By mapping the sequencing data to a reference genome and performing GC correction on the mapping result, the dosage of each chromosome was estimated. These methods are based on that Y chromosome fragments can only come from a male fetus: the higher the FF, the higher the dosage of the Y chromosome; similarly, a male fetus has only one X chromosome, the higher the FF, the lower the X chromosome dosage. Thus, it was possible to infer FF of a male fetus by means of the dosages of sex chromosomes.

(3) Whole-Genome NGS (Paired-End (PE) Sequencing), FF Inference Based on cfDNA Fragment Length Distribution

According to these methods, PE sequencing methods had to be used, so as to infer the lengths of cfDNA fragments according to the mapping positions of Read1 and Read2. These methods are based on the difference between the length distribution of the fetal cfDNA and that of the maternal cfDNA. Studies showed that the length of cfDNAs in the plasma is peaked at 166 bp, and there is another peak at 143 bp, and lower peaks decreasing with a 10 bp periodicity. At a higher FF, in maternal peripheral plasma: the abundance of cfDNAs segments around 143 bp increases, and the abundance of cfDNAs segments around 166 bp decreases. Thus, it was possible to infer the FF according to the cfDNA fragment length distribution in the maternal peripheral plasma.

(4) Targeted NGS Sequencing Methods, Performing High-Depth Sequencing on a Number of SNPs

These methods were able to use a targeted NGS sequencing method to perform high-depth sequencing on a number of SNPs of the maternal peripheral plasma, cfDNAs in the maternal peripheral plasma on said SNPs were deemed as composite genotypes (AAAA, AAAB, ABAA, and ABAB; in each group, the two former letters representing the maternal genotype, and the two latter letters representing the fetal genotype), and the FF was directly estimated according to heterozygosity ratios of these SNPs.

(5) Methylation-Based Methods

These methods are based on the difference of methylation level between the fetal and maternal cfDNAs, and bisulfite sequencing was used to distinguish the cfDNAs of fetal and maternal origin, so as to infer FF.

(6) Methods Based on the Non-Uniformity of Coverage of the Fetal cfDNA of the Whole Genome

These methods are based on that the cfDNA of a gene with high expression level is easier to degrade, the cfDNA of a fetus is derived from the placenta, and a placenta exhibits specific gene expression pattern. Samples of male fetus pregnancy were used to establish a statistical model that used whole-genome coverage data (summarized in bins) to inferring FF.

Accurate quantification of FF has always been a technical challenge, and there are difficulties in many aspects. Traditional FF quantification methods based on sex chromosomes have a disadvantage of not being able to quantify the FF of a female fetus. FF quantification methods based on the length difference between fetal and maternal cfDNA fragments require PE sequencing, increasing sequencing cost, and the accuracy is low. FF quantification methods based on the allele frequencies of SNPs require capture and high-depth sequencing. The experimental processing procedures of methylation-based FF quantification are complicated, rendering higher sequencing cost. Methods based on the non-uniformity of coverage of the fetal cfDNA of the whole genome are not accurate enough.

Hence, there are difficulties in all the available methods, mainly in respect of: additional experiments, requirements for additional instruments and devices, being limited to measurement of male fetuses, low accuracy, and high cost.

For the problems in the prior art, no solution has been provided as yet.

### SUMMARY

The embodiments of the disclosure provide a method and a device for acquiring the FF of cfDNA, a storage medium and an electronic device, so as to solve the problem of high cost of FF measurement in the prior art.

According to one embodiment of the disclosure, a method for acquiring the FF of cfDNA is provided, the method comprising: acquiring sequencing data of a sample taken from a mother pregnant with a fetus; establishing a joint probability distribution model of the maternal and fetal genotypes, the joint probability distribution model containing one or more factors affecting the read heterozygosity, the percentage of the read heterozygosity being the ratio of the number of SNPs covered by different bases to the total number of SNPs covered by more than one reads in the sequencing data; and substituting the values of the one or more factors and of the acquired read heterozygosity into the joint probability distribution model; and obtaining the FF of cfDNA by maximum likelihood estimation of the joint probability distribution model.

Further, in cases where the one or more factors include at least one of: the maternal inbreeding coefficient, the fetal inbreeding coefficient, the sequencing error rate, and the population allele frequency information. The value of the one or more factors is acquired before substituting the values of the one or more factors and of the read heterozygosity into the joint probability distribution model.

Further, in cases where the one or more factors include the maternal inbreeding coefficient, the maternal inbreeding coefficient is acquired by low-depth sequencing of maternal leukocytes, or is acquired together with the FF of cfDNA by maximum likelihood estimation of the joint probability distribution model.

Further, in cases where the one or more factors include the fetal inbreeding coefficient, the fetal inbreeding coefficient is obtained by setting the fetal inbreeding coefficient as 0, or by sequencing of paternal leukocyte, or by using an average value of the population inbreeding coefficients as the fetal inbreeding coefficient.

Further, in cases where the one or more factors include the population allele frequency information, the population allele frequency information is obtained from data of the population to which the mother genetically belongs or is obtained by calculation based on a preset number of NIPS samples.

Further, acquiring the sequencing data of the sample comprises: acquiring raw sequencing data by extracting cfDNA from the sample and performing sequencing; and processing the raw sequencing data to obtain the sequencing data, the processing being used for enabling the raw sequencing data to be suitable for acquiring the read heterozygosity.

Further, processing the raw sequencing data to obtain the sequencing data comprises: removing low-quality reads; and mapping the remaining reads to a reference genome, so as to obtain reads meeting a mapping strategy as the sequencing data.

Further, the low-quality reads include at least one of: duplicated reads introduced by PCR amplification, reads containing one or more bases N, and reads harboring 5 continuous nucleotides with average sequencing quality lower than 20; and/or the mapping strategy comprises allowing at most one mismatch or only maintaining uniquely mapped reads.

Further, extracting cfDNA from the sample and performing sequencing comprises: extracting cfDNA from the sample and performing whole-genome low-depth sequencing.

Further, the joint probability distribution model is described by the following formulas:

| MMFF | Prob | f_{A} |
|---|---|---|
| AA + AA | p³(1 + q/p F₁)(1 + q/p F₂) | 1 − e |
| AB + AB | pq(1 − F₁)(1 − F₂) | 1/2 |
| BB + BB | q³(1 + p/q F₁)(1 + p/q F₂) | e |
| AA + AB | p²q(1 + q/p F₁)(1 − F₂) | (1 − h/2) − (1 − h)e |
| BB + AB | pq²(1 + p/q F₁)(1 − F₂) | h/2 + (1 − h)e |
| AB + AA | p²q(1 − F₁)(1 + q/p F₂) | 1/2 + h/2 (1 − e) |
| AB + BB | pq²(1 − F₁)(q + p/q F₂) | 1/2 − h/2 (1 − e), |

wherein column MMFF shows the maternal and fetal genotypes, A and B respectively represent two alleles at one SNP, column Prob shows a joint probability of the maternal and fetal genotypes, p and q respectively represent the population allele frequency information of the alleles A and B, F1 represents the maternal inbreeding coefficient, F2 represents the fetal inbreeding coefficient, e represents the sequencing error rate, column f_{A} shows the frequency of the allele A in the sequencing data of the sample, and h represents the FF of cfDNA.

According to another embodiment of the disclosure, a device for acquiring the FF of cfDNA is also provided, the device being used for storing or running a module, or a module is a component of the device, wherein the module is a software module, and there is one or more software modules, the software module being used for executing any one of the methods above.

According to another embodiment of the disclosure, a storage medium is also provided, a computer program being stored in the storage medium, wherein the computer program is provided to execute, during running, the steps in any one of the method embodiments above. According to another embodiment of the disclosure, an electronic device is also provided, the electronic device comprising a memory and a processor; a computer program is stored in the memory, and the processor is provided to run the computer program so as to execute the steps in any one of the method embodiments above.

In the embodiments of the disclosure, the provided method for acquiring the FF of cfDNA is used to obtain the FF of cfDNA by establishing a joint probability distribution model of the maternal and fetal genotypes and performing calculation using the values of the factors in the joint probability distribution model and the value of read heterozygosity affected by said factors. Said method is able to use NIPS conventional low-depth NGS data to complete quantitative measurement of the FF without increasing any additional experimental and sequencing cost. Said method requires low cost and provides high accuracy, and is also suitable for FF measurement of female fetuses.

### BRIEF DESCRIPTION OF THE DRAWINGS

Drawings described herein are used for providing further understanding to the disclosure, forming a part of the present application; the schematic embodiments of the disclosure and illustrations thereof are used for explaining the disclosure, without forming improper limitation to the disclosure. In the drawings:

FIG. 1 is a flow chart of a method for acquiring the FF of cfDNA according to embodiment 1 of the disclosure;

FIG. 2 is a diagram of the comparison result of the FF actually acquired based on simulation mixing data of the disclosure with an expected the FF according to embodiment 2;

FIG. 3 is a diagram of the comparison result of the FF acquired based on a real mixed sample with the FF achieved by mixing according to embodiment 3 of the disclosure;

FIG. 4 is a diagram of the comparison result of the FF acquired based on a real male fetus NIPS sample with the FF inferred from a sex chromosome according to embodiment 4 of the disclosure;

FIG. 5 is a diagram of the comparison result of joint-inferred maternal inbreeding coefficient and the FF with the FF inferred based on a sex chromosome according to embodiment 5 of the disclosure;

FIG. 6 is a structural diagram of a device for acquiring the FF of cfDNA according to embodiment 6 of the disclosure; and

FIG. 7 is a detailed structural diagram of a device for acquiring the FF of cfDNA according to embodiment 6 of the disclosure.

### DETAILED DESCRIPTION OF THE EMBODIMENTS

In order to make those skilled in the art better understand the solutions of the disclosure, the technical solutions in the embodiments of the disclosure are clearly and completely described below in combination with the drawings in the embodiments of the disclosure, apparently, the described embodiments are only some, not all, of the embodiments of the disclosure. All the other embodiments obtained by those of ordinary skill in the art on the basis of the embodiments of the disclosure without inventive efforts shall fall within the scope of protection of the disclosure.

It is to be noted that terms ‘include’ and ‘have’ and any variations thereof in the description and claims and the drawings of the disclosure are intended to mean non-exclusive ‘comprising’, for example, a process, a method, a system, a product or a device comprising a series of steps or units is not necessarily limited to the clearly listed steps or units, but other steps or units which are not clearly listed or inherent to said process, method, product or device can be included.

It is to be noted that terms ‘first’, ‘second’ and the like in the description and claims and the drawings of the disclosure are used for distinguishing similar objects, but not necessarily used for describing a specific sequence or a precedence order.

### Embodiment 1

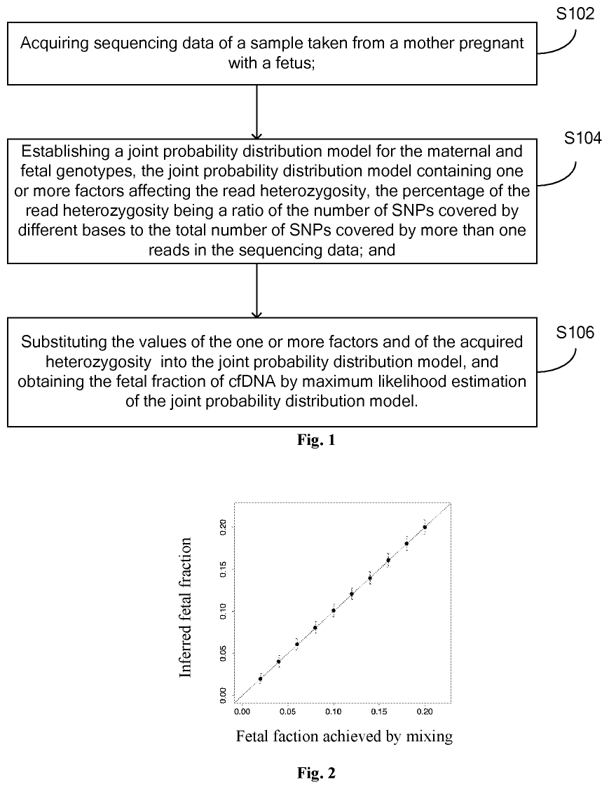

In the present embodiment, a method for acquiring the FF of cfDNA is provided. FIG. 1 is a flow chart of the method for acquiring the FF of cfDNA according to an embodiment of the disclosure. As shown in FIG. 1, the method comprises the steps of:

S102, acquiring sequencing data of a sample taken from a mother pregnant with a fetus;

S104, establishing a joint probability distribution model of the maternal and fetal genotypes, the joint probability distribution model containing one or more factors affecting the read heterozygosity, the percentage of the read heterozygosity being the ratio of the number of SNPs covered by different bases to the total number of SNPs covered by more than one reads in the sequencing data;

S106, substituting the values of the one or more factors and of the acquired read heterozygosity into the joint probability distribution model, and obtaining the FF of cfDNA by maximum likelihood estimation of the joint probability distribution model.

The described method for acquiring the FF of cfDNA is used to obtain the FF of cfDNA by establishing a joint probability distribution model of the maternal and fetal genotypes and performing maximum likelihood estimation using the values of the factors in the model and the value of the read heterozygosity affected by these factors. The method is able to use NIPS conventional NGS low-depth sequencing data to complete quantitative measurement of the FF without introducing any additional experimental and increasing sequencing cost. Said method requires low cost and provides high accuracy, and is also suitable for FF measurement of female fetuses.

Optionally, the executive subject of the described steps can be, but not limited to, a base station, a terminal or the like.

In a preferred embodiment, in cases where the one or more factors include at least one of: the maternal inbreeding coefficient F1, the fetal inbreeding coefficient F2, the sequencing error rate e, and the population allele frequency information, the described method further comprises acquiring the value of the one or more factors before substituting the values of the one or more factors and of the acquired read heterozygosity into the joint probability distribution model.

In practical applications, based on different sequencing data sources, numbers of the described factors affecting the read heterozygosity are not equal, and the values of the factors are also different. For example, in the case of a high sequencing quality, the sequencing error rate e is generally about 0.001. The population allele frequency information is different among different populations, for example, the population allele frequency information acquired from East Asian populations is different from the population allele frequency information acquired from Europe and America populations. Both the maternal inbreeding coefficient F1 and the fetal inbreeding coefficient F2 affect the percentage of read heterozygosity in the sequencing data. The higher the inbreeding coefficients, the lower the probability of read heterozygosity occurs in the fetus; the lower the inbreeding coefficients, the higher the probability of read heterozygosity occurs in the fetus.

In a preferred embodiment, in cases where the one or more factors includes the maternal inbreeding coefficient F1, the maternal inbreeding coefficient F1 can be acquired by low-depth (0.1× to 0.5×) sequencing of maternal leukocyte. Specifically, the maternal inbreeding coefficient F1 can be acquired by establishing a model similar to that of the present application by low-depth sequencing of maternal leukocyte, and setting the fetal fraction h in the model as 0. Otherwise, the maternal inbreeding coefficient F1 can be acquired together with the FF by maximum likelihood estimation of the joint probability distribution model using the cfDNA low-depth sequencing data.

In a preferred embodiment, in cases where the one or more factors include the fetal inbreeding coefficient F2, the fetal inbreeding coefficient F2 is obtained by setting the fetal inbreeding coefficient F2 as 0; or by sequencing of paternal leukocyte; or by using an average value of the population inbreeding coefficients as the fetal inbreeding coefficient.

The fetal inbreeding coefficient F2 is theoretically affected by both the mother and father, and thus theoretically requires sequencing of the paternal leukocyte. However, the inventor of the present application discovered that setting the fetal inbreeding coefficient F2 as 0 or using an average value of the population inbreeding coefficients is sufficient for acquiring the FF of cfDNA, because the mean FF of cfDNA is generally about 10%.

In a preferred embodiment, in cases where the one or more factors include the population allele frequency information, the population allele frequency information is obtained from data of the population to which the mother is subordinate, or by calculation based on a preset number of NIPS samples.

If the population allele frequency information is obtained from the data of the population to which the mother is subordinate, for example, the mother is subordinate to East Asian (ESA), the population allele frequency information can be acquired from East Asian population data of 1000 Genomes Project and Genome Aggregation Database (gnomAD). If the population allele frequency information is acquired by calculation based on a preset number of NIPS samples, for example, the population allele frequency information can be acquired by calculation based on a lot of real NIPS samples, the specific number of the samples can be thousands or tens of thousands.

In the described method, available steps can be used for acquiring the sequencing data of the sample. In a preferred embodiment, acquiring the sequencing data of the sample comprises: obtaining raw sequencing data by extracting cfDNA from the sample and performing sequencing; and processing the raw sequencing data to obtain the sequencing data, the processing being used for processing the raw sequencing data to obtain the sequencing data suitable for acquiring the read heterozygosity.

The specific processing method is similar to an available raw sequencing data processing method, and both methods comprise a step of filtering the raw data to obtain the sequencing data, that is, raw data is processed into clean data. In a preferred embodiment, processing the raw sequencing data to obtain the sequencing data comprises: removing low-quality reads; and mapping the remaining reads to a reference genome, so as to obtain the reads meeting mapping strategy as the sequencing data.

“Low-quality” herein has the same meaning as that of “low-quality” in the conventional high-throughput sequencing field, and it generally refers to the data which may not be subjected to effective data processing, or has apparently adverse effects on the processing result. In a preferred embodiment, the low-quality reads include at least one of: duplicated reads introduced by PCR amplification, reads containing one or more bases N, and reads harboring 5 continuous nucleotides with average sequencing quality lower than 20; and/or the mapping strategy comprises allowing at most one mismatch or only maintaining uniquely mapped reads.

In the described preferred embodiment, bases N means that there may be bases not detectable from the raw data for sequencing, and are represented by N. The sequencing quality can be measured by many available software packages, and thus it is easy to screen out the reads harboring 5 continuous nucleotides with average sequencing quality lower than 20.

In the mapping strategy, at most one mismatch is allowed so as to ensure high quality of the sequencing data for follow-up processing; it is more likely to be caused by a real base type rather than a sequencing error, and thus is helpful to make the FF of cfDNA be more accurate. Only maintaining uniquely mapped reads means that the data for following analysis are the reads which can be completely mapped to the reference genome, so that the detected base type of each SNP is ensured to be real. The amount of data subjected to specific mapping is not limited, and can be rationally set according to different sample sources. Preferably, the processed sequencing data have at least 4M reads.

As for extracting cfDNA from the sample and performing sequencing, conventional sequencing methods in prior art can be used, without the requirement of high-depth sequencing or PE sequencing, and 0.1×-0.5× low-depth sequencing of the available NIPS method can meet the requirements. Of course a high-depth sequencing method also meets the requirements. In a preferred embodiment, extracting the cfDNA from the sample and performing sequencing comprises extracting cfDNA from the sample and performing whole-genome low-depth sequencing. An aimed coverage of 0.1× to 0.5× is enough for the low-depth sequencing herein.

In the described method, the theoretical basis for establishing the joint probability distribution model of the maternal and fetal genotypes is: even in the data of the low-depth sequencing method like NIPS, there are enough 1000 Genomes Project SNPs that are covered by more than one read, and the coverage of these 1000 Genomes Project SNPs conforms to Poisson distribution.

For any SNP covered by more than one read, the SNP can be defined as read homozygosity or read heterozygosity.

There is a functional relationship between the percentage of the read heterozygosity (the number of read heterozygosity accounting for the total SNPs) and the fetal fraction h. As a paternal DNA may be introduced by the fetus, some homozygous SNPs in the sample are changed into heterozygous SNPs. As a low-depth sequencing method is used, the probability that the read heterozygosity can be detected is related to the h. For the same maternal background (F1), the larger the h, the higher the proportion of the detected read heterozygosity. Therefore, the fetal fraction h can be inferred using the percentage of read heterozygosity (the number of read heterozygosity accounting for the total SNPs).

Under the most ideal conditions, it is assumed that both the maternal and fetal inbreeding coefficients are 0, the sequencing error rate of the sequencing platform is also 0, and the population allele frequency is subordinated to the homogeneous distribution, and then the joint probability distribution model for the maternal and fetal genotypes can be obtained, as shown in Table 1 below.

TABLE 1

| MMFF | Prob | f_{A} |
|---|---|---|
| AA + AA | p³ | 1 |
| AB + AB | p(1 − p) | 1/2 |
| BB + BB | (1 − p)³ | 0 |
| AA + AB | p²(1 − p) | 1 − h/2 |
| BB + AB | p(1 − p)² | h/2 |
| AB + AA | p²(1 − p) | 1/2 + h/2 |
| AB + BB | p(1 − p)² | 1/2 − h/2 |

In Table 1, MMFF represents the maternal and fetal genotypes, A and B represent alleles at one SNP, column Prob shows the probability of the corresponding maternal and fetal genotypes, and f_{A} represents the frequency of the allele A in the sequencing data.

If, for some sequencing SNPs, the coverage is 2 and the population allele frequency is p, the percentage of read heterozygosity is as follows:

P_{H}=(1+h−h²)p(1−p)

Integral operation of P_{H} is performed according to p˜uniform(0,1). Under all allele frequencies in the sequencing data, the percentage of read heterozygosity is as follows: ⅙(1+h−h²).

However, in the practical application, the heterozygosity degree can be affected by three factors: the fetal inbreeding coefficient F2, the maternal inbreeding coefficient F1, and the sequencing error rate e.

For the SNPs of two alleles, the inbreeding coefficient F1 directly affects the frequencies of homozygous AA and BB, and heterozygous AB, as follows:

AA˜p²+pqF₁, AB˜2pq(1−F₁), BB˜q²+pqF₁.

Thus, in a preferred embodiment, the joint probability distribution model is as shown in Table 2 below.

TABLE 2

| MMFF | Prob | f_{A} |
|---|---|---|
| AA + AA | p³(1 + q/p F₁)(1 + q/p F₂) | 1 − e |
| AB + AB | pq(1 − F₁)(1 − F₂) | 1/2 |
| BB + BB | q³(1 + p/q F₁)(1 + p/q F₂) | e |
| AA + AB | q²q(1 + q/p F₁)(1 − F₂) | (1 − h/2) − (1 − h)e |
| BB + AB | pq²(1 + p/q F₁)(1 − F₂) | h/2 + (1 − h)e |
| AB + AA | p²q(1 − F₁)(1 + q/p F₂) | 1/2 + h/2 (1 − e) |
| AB + BB | pq²(1 − F₁)(q + p/q F₂) | 1/2 − h/2 (1 − e), |

wherein column MMFF shows the maternal and fetal genotypes, A and B respectively represent two alleles at one SNP, column Prob shows a joint probability of the maternal and fetal genotypes, p and q respectively represent the population allele frequency information of the alleles A and B, F1 represents the maternal inbreeding coefficient, F2 represents the fetal inbreeding coefficient, e represents the sequencing error rate, column f_{A} shows the frequency of the allele A in the sequencing data of the sample, and h represents the FF of cfDNA.

The model can solve h by a maximum likelihood method. The precondition for the solution is that F1, F2, e and the population allele frequency information should be known. Herein, the maternal inbreeding coefficient F1 can be obtained by low-depth sequencing of maternal leukocyte, and the model can be regarded as a special case of a conventional model while h=0. The model can simultaneously solve h and F1 by the maximum likelihood method. In this way, the precision of h is slightly sacrificed, but the cost for maternal leukocyte sequencing is saved. The sequencing error rate e of the platform can be directly obtained from the data. Although theoretically the fetal inbreeding coefficient F2 requires sequencing of the paternal leukocyte, the requirement is satisfied enough in actual operation by letting F2=0 or the average value of the population inbreeding coefficients, as the FF is generally small and is about 10%. The population allele frequency information can be directly acquired from the East Asian population data of 1000 Genomes Project or Genome Aggregation Database (gnomAD), or can be obtained by calculation based on a lot of the real NIPS samples.

Based on the mapped data, substituting the heterozygosity and the homozygosity of a lot of SNPs (the depth being 2 or 3) in autosomes, and the maternal inbreeding coefficient and the population frequency of the SNPs obtained from 1000 Genomes Project data into the actual model, the fetal fraction h of cell-free nucleic acid can be solved.

The low-depth sequencing referred to in the present application means that the coverage of the whole sample is 0.1× to 0.5×. The coverage being 2 or 3 refers to the depth of some SNPs. For example, there are 30 million SNPs in human genome, the depth of some SNPs is 0, the depth of some SNPs is 1, the depth of some SNPs is 2, and there might be certain differences among similar depth of other SNPs; but in average, the depth of the whole sample is 0.5×.

On the basis of the description of the embodiment above, those skilled in the art should clearly understand that the method according to the embodiment above can be implemented with the help of software in combination with a necessary common hardware platform, or by means of hardware, but in many cases the former is the preferred method for implementation. Based on such understanding, the technical solutions of the disclosure substantively or parts thereof contributive to the prior art can be embodied in form of a software product, and the computer software product is stored in a storage medium (for example, an ROM/RAM, a magnetic disk, or an optical disk), including a plurality of instructions configured to enable a terminal device (which can be a mobile phone, a computer, a server, or network equipment, etc.) to execute the method according to the embodiments of the disclosure.

Further explanations are provided below in combination with optional embodiments.

### Embodiment 2: Verification by Simulation of Mixed Data

Whole-genome sequencing data of NA12892 (mother) and NA12878 (daughter) from 1000 Genomes Project were selected, reads were mixed according to different gradients of FF (respectively being 2%, 4%, 6%, 8%, 10%, 12%, 14%, 16%, 18%, and 20%), with a coverage of 0.5×.

The inbreeding coefficients of the mother and the daughter were obtained by whole-genome sequencing reads of the mother and the daughter, the sequencing error rate was calculated based on sample reads obtained by mixing, the population allele frequency of each SNP was acquired from East Asian population data from the East Asian 1000 Genomes Project, a percentage of read heterozygosity was obtained by analyzing the reads of the sample obtained by mixing, and the described parameters were substituted into the described joint probability distribution model for calculation, and thus the FF of cfDNA h can be acquired.

The inferred FFs were compared with expectations, and the comparison results are as shown in FIG. 2. It can be seen from FIG. 2 that the FF acquired by the method of the present application was consistent with the expected FF (proportion of mixed reads). The mixed reads of each gradient were repeated for 100 times, the average values (black dots in the figure) and variances (each vertical line represents adding and subtracting a variance) of h were calculated.

### Embodiment 3: Real Mixed Sample

DNAs derived from a mother and a fetus were mixed according to different FFs (the FF was 3%, 5%, 8% and 12% respectively), and low-depth whole-genome sequencing was performed on a sequencing platform, and then the FF was inferred by means of the method provided in the present application.

Specifically, the sequencing depth was 0.1×, the sequencing error rate was 1/1000, the maternal and fetal inbreeding coefficients were respectively obtained by calculation based on the respective DNA sequencing data, the population allele frequency of each site was acquired from East Asian population data of East Asian 1000 Genomes Project, and the percentage of read heterozygosity in the sequencing data of each mixing fraction was acquired from the sequencing data.

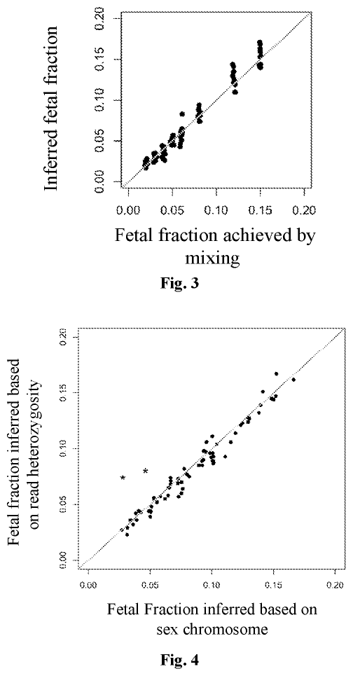

The inferred FF was compared with the FF achieved by mixing, and the comparison results were as shown in FIG. 3. It can be seen from FIG. 3 that the FF acquired by the method was consistent with the FF achieved by mixing.

### Embodiment 4: Verification by Real NIPS Samples with Male Fetuses

69 NIPS clinic samples pregnant with male fetuses were selected, and the FF was acquired by the method of the present application. The inferred FF was compared with that inferred from a sex chromosome. The comparison results were as shown in FIG. 4, and it can be seen from the FIG. 4 that the FF acquired by the method was highly consistent with the FF acquired by the inference method based on the sex chromosomes in 67 samples. As to the two outliers (*), the FF acquired by means of the method of the present application was about twice of the FF inferred on the basis of the sex chromosome. The two samples were opposite-sex twins.

### Embodiment 5: Joint Acquisition of the Maternal Inbreeding Coefficient and the FF

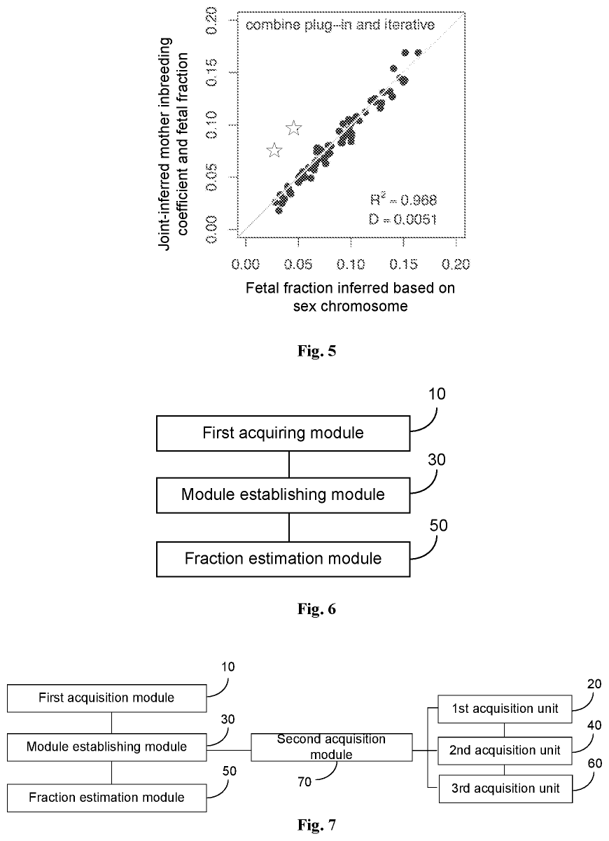

FIG. 5 used the samples same as those in FIG. 4, and the difference was that a maternal inbreeding coefficient and the FF (information of maternal leukocyte was not used) were jointly estimated in FIG. 5. The joint estimation as shown in FIG. 5 can be very accurate, and the samples of opposite-sex twins were presented by asterisks.

It can be seen from the described preferred embodiments that the solutions of the present application have the following advantages:

1) High accuracy: by verification of more than 30 thousand cases of male fetus NIPS samples, the FF acquired by the method of the present application is highly consistent with the FF acquired by the inference method based on sex chromosomes, and R² reaches 98%.

2) Suitable for female fetuses: the problem that the FF of the female fetuses is difficult to be accurately quantified is overcome.

3) Independent of additional experimental procedure and instrument: customized Panel and methylation sequencing are not required, no additional experimental work is required, and the method is independent of any additional experimental instruments or platforms.

4) Low cost, being worthy of wide clinical use. The method of the present application is based on whole-genome low-depth sequencing, and can directly use existing NIPS sample data. PE sequencing is not needed, and high-depth sequencing is not needed (the FF acquisition method based on deep sequencing directly depends on a tiny difference in sequencing depths between two alleles of some heterozygous SNPs, and each heterozygous site needs to be quantitatively analyzed; but the method of the present application calculates the proportion of all heterozygous SNPs accounting for total SNPs, only requiring rough qualitative analysis of heterozygous and homozygous SNPs), and thus does not require additional sequencing cost.

5) Being directly integrable into an NIPS procedure and based on data of NIPS. Thus the method of the present application is easy to be integrated into an NIPS analysis procedure, improving the statistical power of NIPS screening.

### Embodiment 6

Corresponding to the described embodiments, the embodiment further provides a device for acquiring the FF of cfDNA. The device is used for realizing the described embodiments and the preferred embodiments, and what has been explained is not described in detail again. The term ‘module’ used hereafter refers to a combination of software and/or hardware for realizing a preset function. Although the device described in the following embodiments is more optimally implemented in form of software, the implementation of hardware or a combination of both software and hardware is also conceivable.

FIG. 6 is a structural diagram of a device for acquiring the FF of cfDNA according to embodiment 6 of the disclosure. As shown in FIG. 6, the device comprises a first acquisition module 10, a model establishing module 30 and a fraction estimation module 50. In this figure:

the first acquisition module 10 is configured to acquire sequencing data of a sample taken from a mother pregnant with a fetus;

the model establishing module 30 is configured to establish a joint probability distribution model of the maternal and fetal genotypes, the joint probability distribution model containing one or more factors affecting the read heterozygosity, the percentage of the read heterozygosity being the ratio of the number of SNPs covered by different bases to the total number of SNPs covered by more than one reads in the sequencing data; and

the fraction estimation module 50 is configured to supply the values of the one or more factors and of the acquired read heterozygosity into the established joint probability distribution model, and obtain the FF of cfDNA by maximum likelihood estimation of the joint probability distribution model.

By using the device for acquiring the FF of cfDNA, the quantification of the FF of cfDNA is realized without increasing any additional experimental and sequencing cost, and the method requires low cost, provides high accuracy, and is suitable for female FF detection.

FIG. 7 is a detailed structural diagram of a device for acquiring the FF of cfDNA according to embodiment 6 of the disclosure. As shown in FIG. 7, the device comprises all modules as shown in FIG. 6, and further comprises a second acquisition module 70. The second acquisition module is configured to acquire the value of the one or more factors in cases where the one or more factors include at least one of: the maternal inbreeding coefficient, the fetal inbreeding coefficient, the sequencing error rate, and the population allele frequency information.

Optionally, the second acquisition module 70 comprises a first acquisition unit 20 configured to, in cases where the one or more factors include the maternal inbreeding coefficient, acquire the maternal inbreeding coefficient by low-depth sequencing of leukocyte; or by maximum likelihood estimation of the joint probability distribution model leukocyte. Optionally, the second acquisition module 70 comprises a second acquisition unit 40 configured to, in cases where the one or more factors include the fetal inbreeding coefficient, acquire the fetal inbreeding coefficient by setting the fetal inbreeding coefficient as 0; by sequencing of paternal leukocyte; or by using an average value of the population inbreeding coefficients as the fetal inbreeding coefficient.

Optionally, the second acquisition module 70 comprises a third acquisition unit 60 configured to, in cases where the one or more factors include the population allele frequency information, acquire the population allele frequency information from data of the population to which the mother is subordinate; or by calculation based on a preset number of NIPS samples.

Optionally, the first acquisition module 10 comprises: a sample sequencing module configured to acquire raw sequencing data by extracting cfDNA from the sample and performing whole-genome low-depth sequencing; and a processing module configured to process the raw sequencing data to obtain the sequencing data, the processing being used for processing the raw sequencing data to obtain sequencing data suitable for acquiring the read heterozygosity.

Optionally, the processing module comprises: a removing module configured to remove low-quality reads; and a mapping module configured to map the remaining reads to a reference genome, and acquire the reads meeting a mapping strategy as the sequencing data.

Specifically, the low-quality reads include at least one of: duplicated reads introduced by PCR amplification, reads containing one or more base N, and reads harboring 5 continuous nucleotides with average sequencing quality lower than 20; and/or the mapping strategy comprises allowing at most one mismatch or only maintaining uniquely mapped reads.

Optionally, the sample sequencing module comprises a whole-genome low-depth sequencing module configured to extract cfDNA from the sample and perform whole-genome low-depth sequencing.

Optionally, the joint probability distribution model is described by the following formulas:

| MMFF | Prob | f_{A} |
|---|---|---|
| AA + AA | p³(1 + q/p F₁)(1 + q/p F₂) | 1 − e |
| AB + AB | pq(1 − F₁)(1 − F₂) | 1/2 |
| BB + BB | q³(1 + p/q F₁)(1 + p/q F₂) | e |
| AA + AB | p²q(1 + q/p F₁)(1 − F₂) | (1 − h/2) − (1 − h)e |
| BB + AB | pq²(1 + p/q F₁)(1 − F₂) | h/2 + (1 − h)e |
| AB + AA | p²q(1 − F₁)(1 + q/p F₂) | 1/2 + h/2 (1 − e) |
| AB + BB | pq²(1 − F₁)(q + p/q F₂) | 1/2 − h/2 (1 − e), |

wherein column MMFF shows the maternal and fetal genotypes, A and B respectively represent two alleles at one SNP, column Prob shows a joint probability of the maternal and fetal genotypes, p and q respectively represent the population allele frequency information of the alleles A and B, F1 represents the maternal inbreeding coefficient, F2 represents the fetal inbreeding coefficient, e represents the sequencing error rate, column f_{A} shows the frequency of the allele A in the sequencing data of the sample, and h represents the FF of cfDNA.

It is to be noted that the described modules can be implemented by means of software or hardware; the latter can be implemented in the way of, but not limited to: providing the described modules in same processor; or providing the described modules respectively in different processors in form of arbitrary combinations.

### Embodiment 7

The embodiment of the disclosure further provides a storage medium, in which a computer program is stored, wherein the computer program is provided to execute, during running, the steps in any one of the described method embodiments.

Optionally, in this embodiment, the described storage medium can be provided to store the computer program used for executing the following steps:

S1. acquiring sequencing data of a sample taken from a mother pregnant with a fetus;

S2. establishing a joint probability distribution model of the maternal and fetal genotypes, the joint probability distribution model containing one or more factors affecting the read heterozygosity, the percentage of the read heterozygosity being the ratio of the number of SNPs covered by different bases to the total number of SNPs covered by more than one reads in the sequencing data; and

S3. substituting the values of the one or more factors and of the acquired read heterozygosity into the joint probability distribution model; and obtaining the FF of cfDNA by maximum likelihood estimation of the joint probability distribution model.

Optionally, in the embodiment, the described storage medium may comprise, but not limited to, various mediums capable of storing the computer program, such as a USB disk, a read-only memory (ROM), a random access memory (RAM), a mobile hard disk, a magnetic disk or an optical disk.

### Embodiment 8

The embodiment of the disclosure also provides an electronic device comprising a memory and a processor, a computer program being stored in the memory, and the processor being provided for executing the steps in any one of the described method embodiments by running the computer program.

Optionally, the described electronic device can further comprise a transmitting device and an input-output device, the transmitting device being connected with the described processor, and the input-output device being connected with the described processor.

Optionally, in this embodiment, the described processor can be provided to execute the following steps by means of the computer program:

S1. acquiring sequencing data of a sample taken from a mother pregnant with a fetus;

S2. establishing a joint probability distribution model of the maternal and fetal genotypes, the joint probability distribution model containing one or more factors affecting the read heterozygosity, the percentage of the read heterozygosity being the ratio of the number of SNPs covered by different bases to the total number of SNPs covered by more than one reads in the sequencing data; and

S3. substituting the values of the one or more factors and of the acquired read heterozygosity into the joint probability distribution model; and obtaining the FF of cfDNA by maximum likelihood estimation of the joint probability distribution model.

Optionally, for specific examples of this embodiment, reference can be made to the examples described in the embodiments above and optional embodiments, and no more details are provided herein in this embodiment.

Apparently, those skilled in the art should understand that the modules or steps of the disclosure can be realized by means of a common computing device, they can be integrated on a single computing device, or distributed in a network formed by multiple computing devices; optionally, they can be realized by means of executable program codes of the computing device, and thus can be stored in a storage device and executed by the computing device, and in some cases, the shown or described steps can be executed in a sequence different from the described sequence, or they are respectively made into integrated circuit modules, or multiple modules or steps are made into a single integrated circuit module. Thus, the disclosure is not limited to any specific hardware and software combinations.

The above are only the preferred embodiments of the disclosure, it should be indicated that those of ordinary skill in the art can make further improvements and modifications without departing from a principle of the disclosure, and these improvements and modifications should also be deemed as falling within the scope of protection of the disclosure.

What is claimed is:

1. A method for acquiring the fetal fraction of cfDNA, comprising:
acquiring sequencing data of a sample taken from a mother pregnant with a fetus;
establishing a joint probability distribution model of the maternal and fetal genotypes, the joint probability distribution model containing one or more factors affecting the read heterozygosity, the percentage of the read heterozygosity being the ratio of the number of SNPs covered by different bases to the total number of SNPs covered by more than one reads in the sequencing data; and
substituting the values of the one or more factors and of the acquired read heterozygosity into the established joint probability distribution model; and obtaining the fetal fraction of cfDNA by maximum likelihood estimation of the joint probability distribution model.

2. The method as claimed in claim 1, wherein in cases where the one or more factors include at least one of: the maternal inbreeding coefficient, the fetal inbreeding coefficient, the sequencing error rate, and the population allele frequency information, the value of the one or more factors is acquired before substituting the values of the one or more factors and of the read heterozygosity into the joint probability distribution model.

3. The method as claimed in claim 2, wherein in cases where the one or more factors include the maternal inbreeding coefficient, the maternal inbreeding coefficient is acquired by low-depth sequencing of maternal leukocytes; or the maternal inbreeding coefficient is acquired together with the fetal fraction of cfDNA by maximum likelihood estimation of the joint probability distribution model.

4. The method as claimed in claim 2, wherein in cases where the one or more factors include the fetal inbreeding coefficient, the fetal inbreeding coefficient is obtained by:
setting the fetal inbreeding coefficient as 0; or
sequencing of paternal leukocyte; or
using an average value of the population inbreeding coefficients as the fetal inbreeding coefficient.

5. The method as claimed in claim 2, wherein in cases where the one or more factors include the population allele frequency information, the population allele frequency information is obtained
from data of the population to which the mother genetically belongs; or
by calculation based on a preset number of NIPS samples.

6. The method as claimed in claim 1, wherein acquiring the sequencing data of the sample comprises:
acquiring raw sequencing data by extracting cfDNA from the sample and performing whole-genome low-depth sequencing; and
processing the raw sequencing data to obtain the sequencing data, the processing being used for enabling the raw sequencing data to be suitable for acquiring the read heterozygosity.

7. The method as claimed in claim 6, wherein processing the raw sequencing data to obtain the sequencing data comprises:
removing low-quality reads; and
mapping the remaining reads to a reference genome, and acquiring the reads meeting a mapping strategy as the sequencing data.

8. The method as claimed in claim 7, wherein
the low-quality reads include at least one of: duplicated reads introduced by PCR amplification, reads containing one or more bases N, and reads harboring 5 continuous nucleotides with average sequencing quality lower than 20; and/or
the mapping strategy comprises allowing at most one mismatch or only maintaining uniquely mapped reads.

9. The method as claimed in claim 2, wherein the joint probability distribution model is described by the following formulas:

| MMFF | Prob | f_{A} |
|---|---|---|
| AA + AA | p³(1 + q/p F₁)(1 + q/p F₂) | 1 − e |
| AB + AB | pq(1 − F₁)(1 − F₂) | 1/2 |
| BB + BB | q³(1 + p/q F₁)(1 + p/q F₂) | e |
| AA + AB | p²q(1 + q/p F₁)(1 − F₂) | (1 − h/2) − (1 − h)e |
| BB + AB | pq²(1 + p/q F₁)(1 − F₂) | h/2 + (1 − h)e |
| AB + AA | p²q(1 − F₁)(1 + q/p F₂) | 1/2 + h/2 (1 − e) |
| AB + BB | pq²(1 − F₁)(q + p/q F₂) | 1/2 − h/2 (1 − e), |

wherein column MMFF shows the maternal and fetal genotypes, A and B respectively represent two alleles at one SNP, column Prob shows a joint probability of the maternal and fetal genotypes, p and q respectively represent the population allele frequency information of the alleles A and B, F1 represents the maternal inbreeding coefficient, F2 represents the fetal inbreeding coefficient, e represents the sequencing error rate, column fit shows the frequency of the allele A in the sequencing data of the sample, and h represents the fetal fraction of cfDNA.

10. A device for acquiring the fetal fraction of cfDNA, comprising:
a first acquisition module, configured to acquire sequencing data of a sample taken from a mother pregnant with a fetus;
a model establishing module, configured to establish a joint probability distribution model of the maternal and fetal genotypes, the joint probability distribution model containing one or more factors affecting the read heterozygosity, the percentage of the read heterozygosity being the ratio of the number of SNPs covered by different bases to the total number of SNPs covered by more than one reads in the sequencing data; and
a fraction estimation module, configured to supply the values of the one or more factors and of the acquired read heterozygosity into the established joint probability distribution model; and obtaining the fetal fraction of cfDNA by maximum likelihood estimation of the joint probability distribution model.

11. The device as claimed in claim 10, wherein the device further comprises a second acquisition module, configured to acquire the value of the one or more factors in cases where the one or more factors include at least one of: the maternal inbreeding coefficient, the fetal inbreeding coefficient, the sequencing error rate, and the population allele frequency information.

12. The device as claimed in claim 11, wherein the second acquisition module comprises: a first acquisition unit, configured to, in cases where the one or more factors include the maternal inbreeding coefficient, acquire the maternal inbreeding coefficient by:
low-depth sequencing of maternal leukocytes; or
maximum likelihood estimation of the joint probability distribution model.

13. The device as claimed in claim 11, wherein the second acquisition module comprises: a second acquisition unit, configured to, in cases where the one or more factors include the fetal inbreeding coefficient, acquire the fetal inbreeding coefficient by:
setting the fetal inbreeding coefficient as 0; or
sequencing of paternal leukocyte; or
using an average value of the population inbreeding coefficients as the fetal inbreeding coefficient.

14. The device as claimed in claim 11, wherein the second acquisition module comprises: a third acquisition unit, configured to, in cases where the one or more factors include the population allele frequency information, acquire the population allele frequency information
from data of the population to which the mother genetically belongs; or
by calculation based on a preset number of NIPS samples.

15. The device as claimed in claim 10, wherein the first acquisition module comprises:
a sample sequencing module, configured to acquire raw sequencing data by extracting cfDNA from the sample and performing whole-genome low-depth sequencing; and
a processing module, configured to process the raw sequencing data to obtain the sequencing data, the processing being used for enabling the raw sequencing data to be suitable for acquiring the read heterozygosity.

16. The device as claimed in claim 15, wherein the processing module comprises:
a removing module, configured to remove low-quality reads; and
a mapping module, configured to map the remaining reads to a reference genome, and acquiring the reads meeting a mapping strategy as the sequencing data.

17. The device as claimed in claim 16, wherein
the low-quality reads include at least one of: duplicated reads introduced by PCR amplification, reads containing one or more bases N, and reads harboring 5 continuous nucleotides with average sequencing quality lower than 20; and/or
the mapping strategy comprises allowing at most one mismatch or only maintaining uniquely mapped reads.

18. The device as claimed in claim 10, wherein the joint probability distribution model is described by the following formulas:

| MMFF | Prob | f_{A} |
|---|---|---|
| AA + AA | p³(1 + q/p F₁)(1 + q/p F₂) | 1 − e |
| AB + AB | pq(1 − F₁)(1 − F₂) | 1/2 |
| BB + BB | q³(1 + p/q F₁)(1 + p/q F₂) | e |
| AA + AB | p²q(1 + q/p F₁)(1 − F₂) | (1 − h/2) − (1 − h)e |
| BB + AB | pq²(1 + p/q F₁)(1 − F₂) | h/2 + (1 − h)e |
| AB + AA | p²q(1 − F₁)(1 + q/p F₂) | 1/2 + h/2 (1 − e) |
| AB + BB | pq²(1 − F₁)(q + p/q F₂) | 1/2 − h/2 (1 − e), |

wherein column MMFF shows the maternal and fetal genotypes, A and B respectively represent two alleles at one SNP, column Prob shows a joint probability of the maternal and fetal genotypes, p and q respectively represent the population allele frequency information of the alleles A and B, F1 represents the maternal inbreeding coefficient, F2 represents the fetal inbreeding coefficient, e represents the sequencing error rate, column f_{A} shows the frequency of the allele A in the sequencing data of the sample, and h represents the fetal fraction of cfDNA.

19. A storage medium in which a computer executable program is stored, wherein the method for acquiring the fetal fraction of cfDNA as claimed in claim 1 is executed when the program is set to run.

20. An electronic device, comprising a memory and a processor, wherein a computer program is stored in the memory, and the processor is set to run the computer program so as to execute the method for acquiring the fetal fraction of cfDNA as claimed in claim 1.
